(19) United States
(12) Patent Application Publication
(10) Pub. No.: US 2008/0213297 A1
(43) Pub. Date: Sep. 4, 2008

(54) Method of producing conjugate vaccines

(75) Inventors: Nicholas Finn, Cambridge (GB); Martin Quibell, Cambridge (GB); Manoj Kumar Ramjee, Cambridge (GB); William Gordon Turnell, Delmar, CA (US)

Correspondence Address: EDWARDS ANGELL PALMER & DODGE LLP, P.O. BOX 55874, BOSTON, MA, 02205, US

(73) Assignee: Amura Therapeutics Limited

(21) Appl. No.: 11/879,342

(22) Filed: Jul. 16, 2007

(30) Foreign Application Priority Data
Jan. 18, 2005 (GB) 0501008.7
Jan. 18, 2006 (GB) PCT/GB2006/000160

(51) Int. Cl. A61K 39/385; C07H 1/00; A61P 31/00; C07K 14/00

(52) U.S. Cl. 424/193.1; 536/124; 530/402

(57) ABSTRACT

The present invention relates to a method of production of a hydrazide modified sugar comprising a step of reacting a sugar with a hydrazide in a reaction solvent at a pH of between 3 and 5.5, wherein the solvent comprises an aqueous based solvent and an optional polar organic co-solvent. A further aspect of the invention relates to a method of production of a polysaccharide epitope carrier protein conjugate comprising the steps of: (a) reacting a polysaccharide epitope with a hydrazide to form a hydrazide modified polysaccharide epitope; (b) reacting the hydrazide modified polysaccharide epitope with a linker that has been pre-coupled to a carrier protein. Another aspect of the invention relates to a method of production of a sugar-dihydrazide-aldehyde adduct comprising the steps of: (a) producing a hydrazide modified sugar using a method according to the invention, wherein the hydrazide modified sugar includes a further unreacted hydrazide moiety; and (b) reacting the further hydrazide moiety with the aldehyde functionality of a linker group.

### RELATED APPLICATIONS

This is a utility patent application which claims priority to patent application number PCT/GFB2006/000160, filed on Jan. 18, 2006, which claims priority to GB patent application number 0501008.7, filed on Jan. 18, 2005, the entirety of which is incorporated herein by reference

### BACKGROUND

The emergence of antibiotic resistant microorganisms is well known and a cause of growing concern. Drug resistant strains of, for example, tuberculosis and methicillin resistant Staphylococcus aureus (MRSA) are now common in UK hospitals. The propagation of such resistance could lead to a return of incurable diseases not seen since the last century Vaccination against these infectious agents is an effective and attractive strategy, since vaccines prevent disease, thereby avoiding the use of antibiotics. Vaccination also has the advantages of relatively longer during of action, cheaper costs and better patient compliance.

Protein glycosylation is a complex phenomenon that can involve anywhere from a few carbohydrate residues through to a large branched polysaccharides. Infectious agents such as human immunodeficiency virus (HIV), influenza and encapsulated bacteria express polysaccharide molecules at their surface. These structures serve several functions, but in particular they shield the organism from the patrolling cells of the immune system and enable the infectious agents to evade detection and attack. By training the immune system to recognize these polysaccharide molecules as foreign, through vaccination, the infectious agents can be targeted by a directed immune response.

Currently, vaccines are available to provide protection against bacteria responsible for certain types of pneumonia and meningitis and focus on the capsular polysaccharides (large carbohydrate molecules) found on the surface of these microorganisms. However, despite successfully protecting the adult population, the promise of such sub-unit vaccines has been limited by their low immunogenicity in infants and at risk groups such as the elderly and immuno-compromised individuals. In 1929, Avery and Goebel showed that by conjugating a bacterial polysaccharide to a carrier protein, a stronger immune response could be obtained (Avery, O. T., Goebel, W. F. J. Exp. Med. 50, 533-50, 1929.). Thus, in order to make polysaccharide vaccines broadly more effective, the polysaccharides require conjugation to “carrier proteins”, which are often prepared from bacterial sources. This approach was adopted and resulted in the conjugate vaccines that are available today. The resultant conjugate vaccines tend to be highly immunogenic and confer long lasting protection in most subjects, including infants and children.

Elaborate carbohydrate molecules are also found on many cell surfaces and are often involved in recognition and binding, acting as receptors for other saccharides or proteins. Certain cancers display aberrant glycosylation on their cell surface as a result of malfunctions within the cellular machinery and these carbohydrates are different from those displayed on healthy cells. Using these “tumour associated” rogue molecules in vaccine preparations, which prime the body to mount an immune response against the cancerous cells displaying these “markers”, has proved an attractive strategy and may lead to promising anti-cancer therapies.

In producing a conjugate vaccine it is the step of linking the polysaccharide to the carrier protein which is important because it dictates how these large molecules are recognised by the immune system. Failure to mimic the presentation of the polysaccharide as it appears on the bacterial cell surface greatly diminishes the immunogenic potential of the vaccine. The chemistry employed for the conjugation step should therefore be highly specific and selective and maintain the structural integrity of the polysaccharide, while at the same time allowing simple quality control. The existing conjugation techniques fail to satisfy one or more of these criteria.

Conjugation of polysaccharides to a carrier protein is complicated because of the poly-functional nature and complexity of the molecules. Existing methods often employ cyanogen bromide (CNBr) or 1-cyano-4-dimethylamino pyridinium tetrafluoroborate (CDAP) activation of the carbohydrate, followed by treatment with a homo- or hetero-bifunctional spacer unit (e.g. adipic dihydrazide). This hydrazide modified carbohydrate is then coupled to the carboxyl side chains of the protein using a carbodiimide based reagent (e.g. 1-ethyl-3-[3-dimethylaminopropyl]carbodiimide (EDC)) [Axen, R. et al. Nature, 214(95), 1302-4 1967; Kohn, J. & Wilchek, M. FEBS Lett., 154(1), 209-10, 1983; and Shafer, D. E. et al., Vaccine, 18, 1273-81, 2000]. CNBr and CDAP have a major drawback in that activation through the hydroxyl groups is non-specific and can result in many attachment points being created rather than one specific modification. In addition, using carbodiimides can lead to intra- and inter-molecular cross-linking of the protein, besides the desired reaction with the polysaccharide. CNBr is also highly toxic and requires extremely careful handling, which does not lend itself well to large scale production. Other methods include reductive amination or hydrazone formation at the reducing sugar with amines or hydrazides respectively, but both involve opening the cyclic sugar into its linear form, thus altering the native structural integrity of the carbohydrate/polysaccharide.

In WO 03/087824 the present applicant discloses a technique for conjugating a peptide antigen to a carrier protein. This “AmLinker” technology was developed to allow specific, controlled conjugation between simple and complex molecules, while retaining native structural configuration. The conjugation reaction can be performed in the presence of other functional groups, without the need for complex chemical protection strategies normally required to prevent the occurrence of side reactions. In a typical scenario, where a relevant vaccine candidate (epitope) requires conjugation to a carrier protein, the N-ε-amine of the lysine side chains of the protein are initially modified by acylation with the linking agent, followed by simply stirring with the hydrazide derivatised epitope to form a hydrazone linkage between epitope and protein (Scheme 1). This is the only reaction that can occur when the correct pH conditions are used, resulting in a highly specific and facile conjugation process.

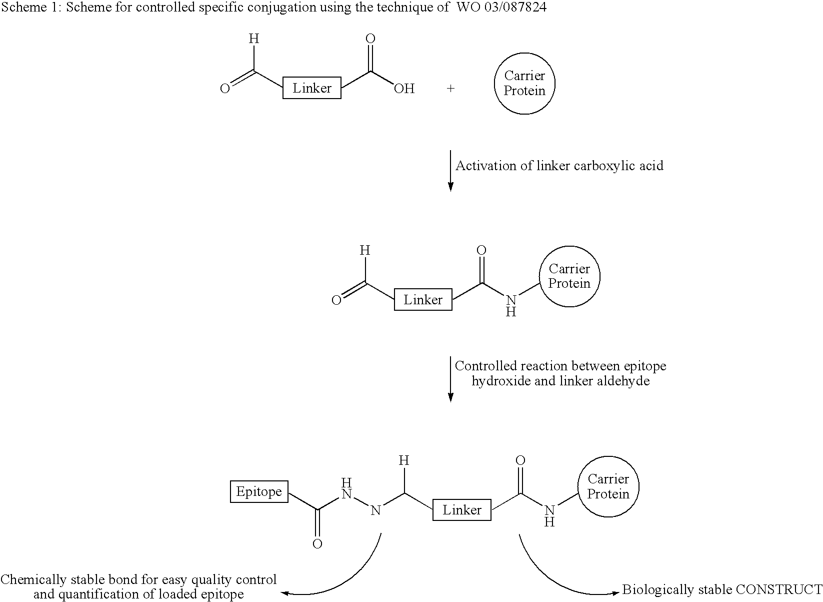

However, controlled coupling of polysaccharide epitopes is less well defined than with peptide epitopes, because sugar chemistry does not easily lend itself to the synthetic strategies available for peptides. There is therefore a need for a technique which allows effective conjugation of a polysaccharide epitope to a carrier protein.

According to the present invention there is provided a method of production of a hydrazide modified sugar comprising a step of reacting a sugar with a hydrazide in a reaction solvent at a pH of between 3 and 5.5, wherein the solvent comprises an aqueous based solvent and an optional polar organic co-solvent.

Preferably, the method does not require the presence of additional coupling agents or activators (for example, carbodiimide based reagents).

Preferably, the method is a simple one-pot process, i.e. which does not require initial conversion to an amino derivative.

The sugar may be a mono-, di- or polysaccharide. Preferably the saccharide is a polysaccharide. Preferably the saccharide is a polysaccharide epitope. Herein, the term “epitope” refers to a molecule which is capable of binding specifically to a biological molecule such as an antibody, antigen or cell surface receptor. The polysaccharide epitope may be an antigenic determinant derived from a surface molecule from a pathogenic organism (such as derived from a surface polysaccharide from a bacteria). The polysaccharide or polysaccharide epitope may be a tumour associated antigen, for example Lewis Y tetrasaccharide. The polysaccharide or polysaccharide epitope may be derived from surface displayed bacterial capsular polysaccharides, (e.g. Streptococcus, Staphylococcus, Neisseria, Pseudomonas) viral glycoproteins (human immunodeficiency virus, respiratory syncitial virus, herpes simplex virus, influenza, rotavirus, papilloma) or tumour associated antigens (Lewis Y, Globo H, melanoma associated ganglioside GM-2, mucin derived Tn and STn antigens) and preferably induces specific immune response when immunised either alone, with an adjuvant or conjugated to a carrier. The saccharide may be a disaccharide, for example α-D-lactose, an aminosugar (for example glucosamine), or an N-acetylamino sugar such as N-acetyl glucosamine.

Preferably, the pH is between 3.5 and 5, more preferably the pH is between 4 and 5, for example a pH value of 4.75. The preferred pH ranges combine good stability with favourable reaction kinetics. Preferably the reaction solvent includes a buffer solution, which maintains the pH within the preferred range or at the preferred value.

The aqueous solvent may be water. Preferably the aqueous solvent is a buffer solution, for example a formate buffer solution. The amount of (optional) polar organic co-solvent is preferably up to 50% (by volume) of the total amount of the reaction solvent, more preferably 10 to 30% (by volume) of the total amount of the reaction solvent. The components of the reaction solvent are chosen according to the other reagents. For example, where the sugar is a polysaccharide of more than 100 kD, a larger proportion of the polar organic co-solvent may be required to aid dissolution of the polysaccharide.

Preferably the hydrazide is a dihydrazide, such as adipic dihydrazide. Preferably the dihydrazide is a branched or straight chain alkyl of up to 10 carbon atoms (preferably four to six carbon atoms) having a first hydrazide moiety at one end of the alkyl chain and the second hydrazide moiety at the other end of the chain. When the hydrazide is a dihydrazide, the hydrazide modified sugar (which is formed by reaction of the sugar with one of the hydrazide functionalities of the dihydrazide) may have an unreacted hydrazide moiety. The reaction conditions may be chosen to maximise this: thus, for example, use of an excess (e.g. up to 10-fold excess, preferably 3 to 5-fold excess) of the (di)hydrazide compared to the sugar should minimise the amount of di-adducts. With the preferred dihydrazides, the unreacted hydrazide moiety or group will be at the opposite end of the alkyl chain to the sugar (and the unreacted hydrazide will be referred to as the “distal hydrazide”). The unreacted hydrazide moiety or group (distal hydrazide) may facilitate further reactions with linkers and binders, as discussed below.

According to the present invention in a still further aspect there is provided a method of production of a polysaccharide epitope carrier protein conjugate comprising the steps of:

(a) reacting a polysaccharide epitope with a hydrazide to form a hydrazide modified polysaccharide epitope;
(b) reacting the hydrazide modified polysaccharide epitope with a linker which is bound to a carrier protein. Preferably the linker has been pre-coupled to a carrier protein.

Preferably the hydrazide in step (a) is a dihydrazide and the product of step (a), the hydrazide modified polysaccharide epitope, includes a further unreacted hydrazide moiety; in this case, step (b) may include the reaction of the further hydrazide moiety with a suitable group on the linker. Preferably reaction (a) and/or reaction (b) is performed in a reaction solvent at a pH of between 3 and 5.5, wherein the solvent comprises an aqueous base solvent and an optional polar organic co-solvent. Preferably, the pH is between 3.5 and 5, more preferably the pH is between 4 and 5. The preferred pH ranges combine good stability with favourable reaction kinetics. Preferably the reaction solvent includes a buffer solution which maintains the preferred range.

As for the above-mentioned first aspect of the invention, preferably, the method does not require the presence of additional coupling agents or activators (for example, carbodiimide based reagents). Preferably, the method is a simple one-pot process, i.e. which does not require initial conversion to an amino derivative.

The “linker” molecule may be any molecule which reacts with the hydrazide modified sugar (the hydrazide modified polysaccharide epitope etc.). With the preferred hydrazides, the dihydrazides, the hydrazide modified sugar (the hydrazide modified polysaccharide epitope etc.) includes a further hydrazide moiety. Preferred linker molecules include a functionality (e.g. an aldehyde functionality) which reacts with the further hydrazide moiety. Preferably the linker is capable of undergoing a specific chemical reaction with both a carrier and the further hydrazide. Preferably the linker molecule is a positive charge balanced linker such as those disclosed in WO03/087824, such as compound 21 herein.

The “carrier” may be a proteinaceous molecule. Examples of suitable carrier proteins include bovine serum albumin (BSA), ovalbumin and keyhole limpet haemocyanin, heat shock proteins (HSP), thyroglobulin, immunoglobulin molecules, tetanus toxoid, purified protein derivative (PPD), aprotinin, hen egg-white lysozyme (HEWL), carbonic anhydrase, ovalbumin, apo-transferrin, l holo-transferrin, phosphorylase B, β-galactosidase, myosin, bacterial proteins and other proteins well known to those skilled in the art. Inactive virus particles (e.g. the core antigen of Hepatitis B Virus, see Murray, K. and Shiau, A-L., Biol. Chem. 380, 277-283, 1999) and attenuated bacteria such as Salmonella may also be used as carriers for the presentation of active moieties.

Preferably, the polysaccharide epitope carrier protein conjugate is a synthetic Le^{y}-BSA conjugate (in which case the polysaccharide epitope is Lewis Y tetrasaccharide; and the carrier protein is BSA).

Preferably, the polysaccharide epitope carrier protein conjugate is, or is suitable for use in, a pharmaceutical composition. Preferably, the pharmaceutical composition is a vaccine composition. The pharmaceutical composition may include a pharmaceutically acceptable adjuvant. In one preferred embodiment, the pharmaceutical composition comprises a pharmaceutically acceptable diluent, excipient or carrier. Examples of suitable excipients may be found in the “Handbook of Pharmaceutical Excipients, 2^{nd} Edition, (1994), Edited by A Wade and P J Weller. Acceptable carriers or diluents for therapeutic use are well known in the pharmaceutical art, and are described, for example, in Remington's Pharmaceutical Sciences, Mack Publishing Co. (A. R. Gennaro edit. 1985).

Thus, according to the present invention in a still further aspect there is provided the use of a hydrazide modified sugar and/or a sugar-dihydrazide-aldehyde adduct and/or a polysaccharide epitope carrier protein conjugate in the manufacture of a diagnostic or a pharmaceutical composition. Preferably the pharmaceutical composition is a vaccine composition.

According to the present invention in a further aspect there is provided a method of production of a sugar-dihydrazide-aldehyde adduct comprising the steps of:

(a) producing a sugar hydrazide adduct by any of the methods above, wherein the sugar hydrazide adduct includes a further unreacted hydrazide moiety; and
(b) reacting the further hydrazide moiety with the aldehyde functionality of a linker group.

Preferably reaction (b) is performed in a reaction solvent at a pH of between 3 and 5.5, wherein the solvent comprises an aqueous base solvent and an optional polar organic co-solvent. At pH below about 3 and above about 5.5 the reactants (e.g. the hydrazide) are unstable. Preferably, the pH is between 3.5 and 5, more preferably the pH is between 4 and 5. The preferred pH ranges combine good stability with favourable reaction kinetics. Preferably the reaction solvent includes a buffer solution, which maintains the preferred range.

The “linker” molecule may be any molecule which reacts with the further hydrazide moiety. Preferred linker molecules include an aldehyde functionality which reacts with the further hydrazide moiety. The reactant aldehyde may be a simple aldehyde such as 2-hydroxy benzaldehyde. Preferably the linker is capable of undergoing a specific chemical reaction with both a carrier and the further hydrazide. Preferably the linker molecule is a positive charge balanced linker as set out above. Preferably the linker is bound to a carrier, as defined above.

As for the above-mentioned first and second aspects of the invention, preferably, the method does not require the presence of additional coupling agents or activators (for example, carbodiimide based reagents). Preferably, the method is a simple one-pot process, i.e. which does not require initial conversion to an amino derivative.

The present methods allow specific modification of a reducing end sugar in a polysaccharide with a bifunctional hydrazide spacer. The reaction is quantitative, performed in an aqueous based solvent, and does not require complicated protection strategies or additional coupling reagents. Conjugation of the so-formed product hydrazide sugar to a linker-modified carrier protein is a simple “add and stir” reaction and can be monitored in situ, in real time, by e.g. absorbance spectroscopy. The reactions may allow the structural conformation of the polysaccharide to remain unchanged throughout the conjugation process (as illustrated by the example below in which the monoclonal antibody raised against Le^{y} on a human cell line is able to recognise a synthetic Le^{y}-BSA conjugate); such retention of structural conformation is particularly important when producing conjugate vaccines. The method may allow high loading of the polysaccharide on a carrier protein (via the bifunctional hydrazide spacer and the linker) while maintaining excellent aqueous solubility. The conjugation reactions are reversible allowing simple characterisation and ease of quality control of the final conjugate, which is also extremely important in vaccine formation.

### DETAILED DESCRIPTION OF THE INVENTION

The present invention will now be described in more detail with reference to the attached figures and the Examples, which are not intended to be limiting.

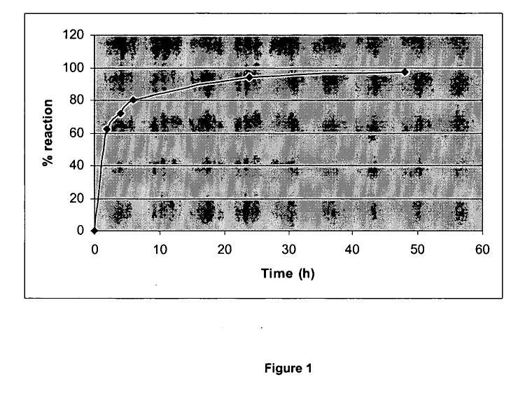

FIG. 1 shows hydrazone formation between benzaldehyde 14 and hydrazide 2 (monitored by NMR);

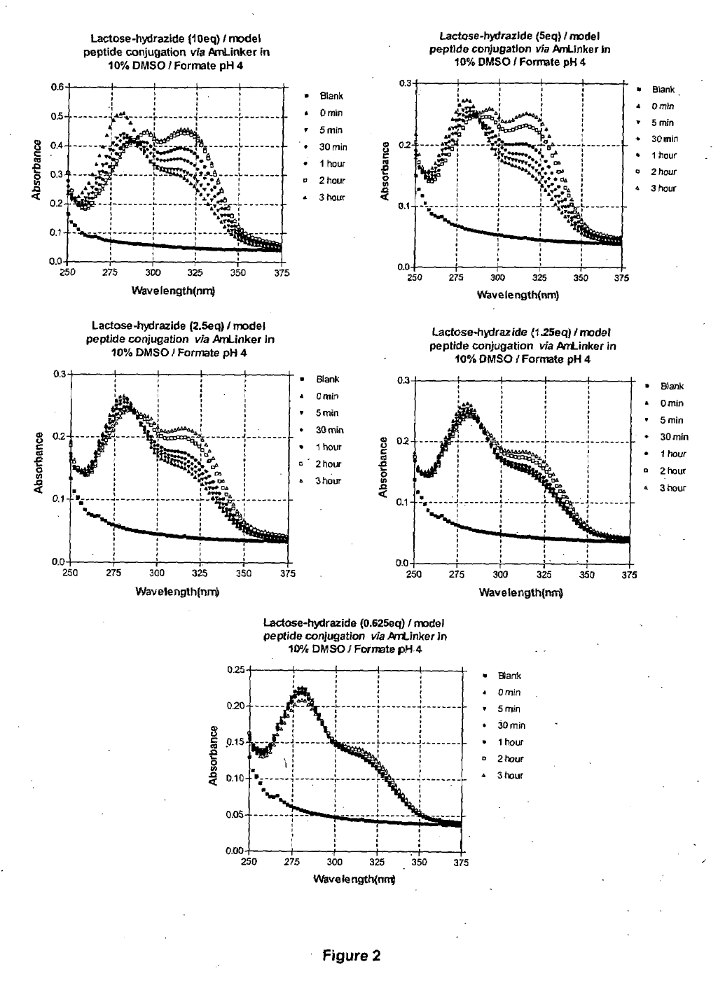

FIG. 2 shows the effect of altering the molar equivalents of lactose hydrazide 6 on the conjugation reaction with AmLinker-peptide 22;

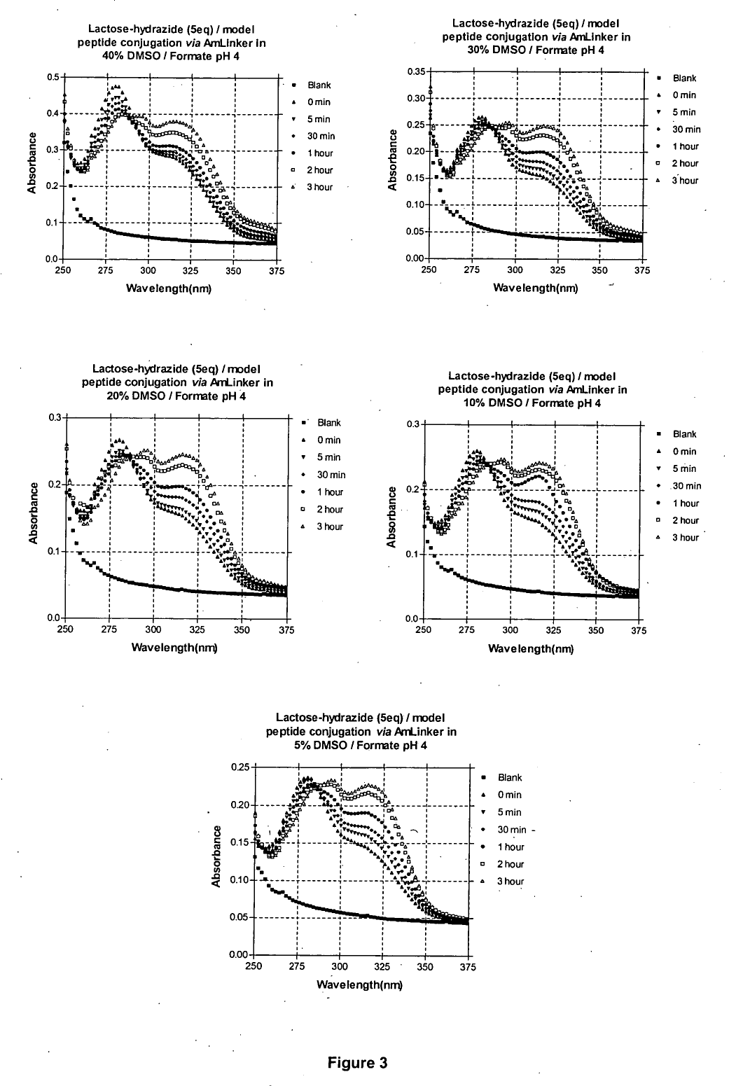

FIG. 3 shows the effect of altering the DMSO concentration on the conjugation reaction with AmLinker-peptide 22 and lactose hydrazide 6;

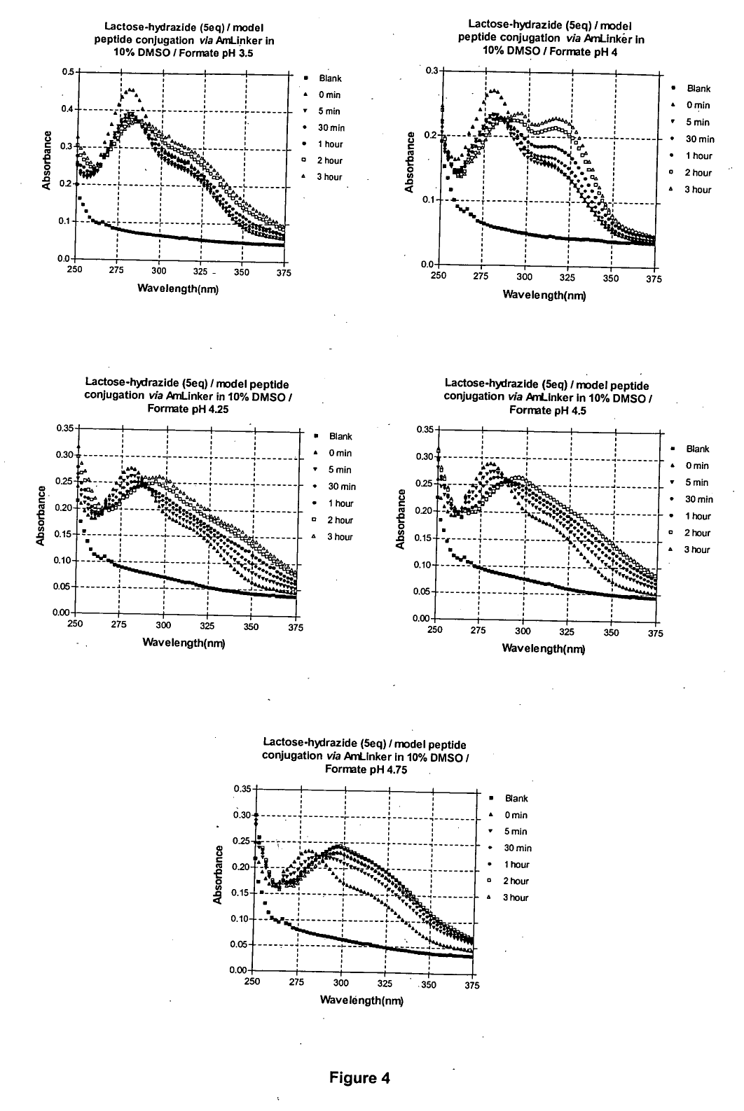

FIG. 4 shows the effect of altering the pH on the conjugation reaction with AmLinker-peptide 22 and Lactose hydrazide 6;

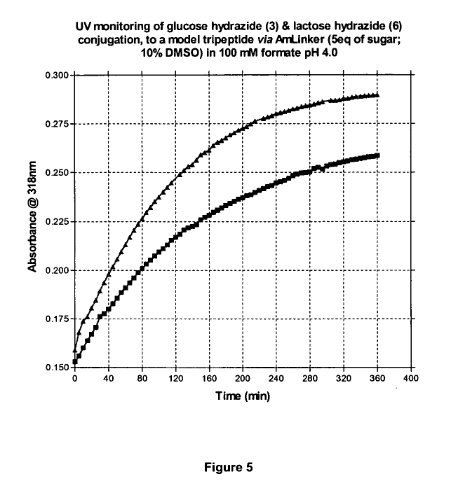

FIG. 5 shows production of conjugates 23 (squares) and 24 (triangles) using the optimal conditions elucidated;

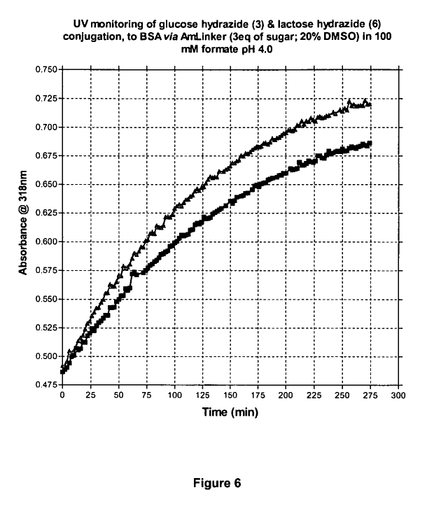

FIG. 6 shows production of BSA-conjugates 25 (squares) and 26 (triangles) of hydrazide sugars 3 and 6 using the optimal conditions elucidated;

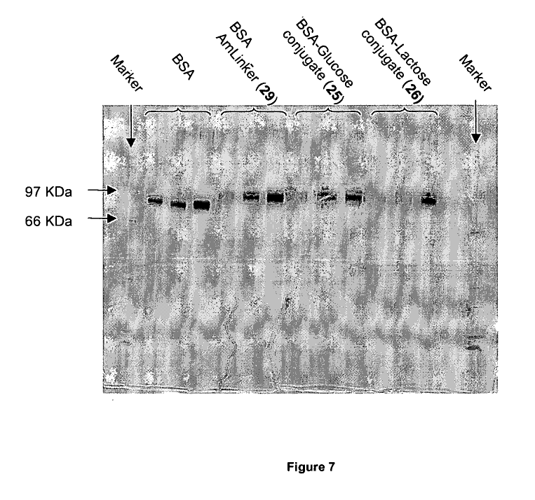

FIG. 7 shows characterisation of conjugates 25 and 26 by gel electrophoresis;

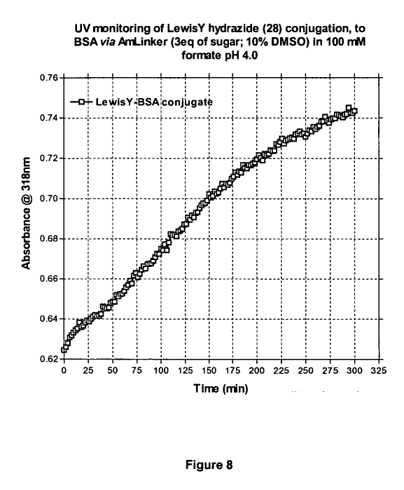

FIG. 8 shows production of Le^{y}-BSA conjugate 30;

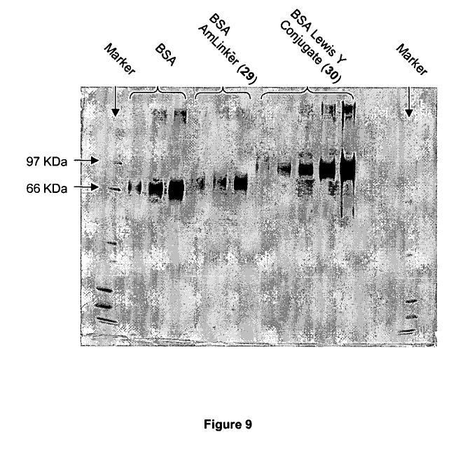

FIG. 9 shows characterisation of Le^{y}-BSA conjugate 30 by gel electrophoresis;

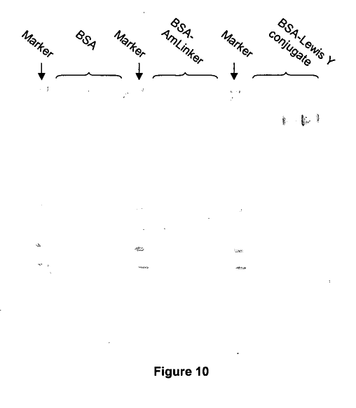

FIG. 10 shows Western Blot of BSA, BSA-AmLinker 29 and BSA-Lewis Y conjugate 30 using a Le^{y} specific mAb and an anti-mouse IgM HRP labelled secondary antibody (visualisation was achieved with 3,3′,5,5′-tetramethylbenzidine (TMB). Rainbow markers were used to give molecular weight estimates);

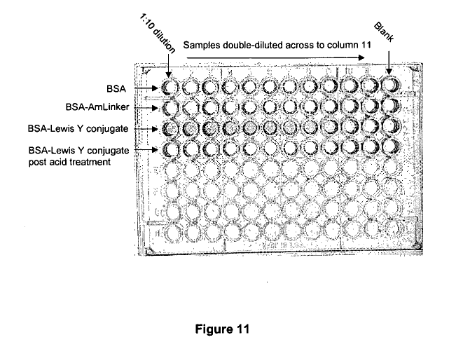

FIG. 11 shows ELISA of BSA, BSA-AmLinker 29 and BSA-Lewis Y conjugate 30 (pre- and post acid treatment) using a Le^{y} specific mAb and an anti-mouse IgM HRP labelled secondary antibody [Visualisation was achieved with o-phenylenediamine (OPD)]; and

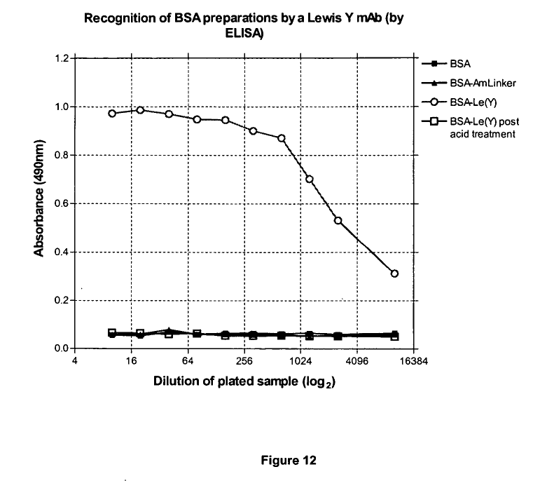

FIG. 12 is a graphical representation of the ELISA seen in FIG. 11 of BSA, BSA-AmLinker 29 and BSA-Lewis Y conjugate 30 (pre- and post acid treatment) using a Le^{y} specific mAb and an anti-mouse IgM HRP labelled secondary antibody [Visualisation was achieved with o-phenylenediamine (OPD)], (quantified by absorbance at 490 nm).

### SYNTHESIS OF HYDRAZIDE MODIFIED SUGARS

An initial experiment showed that hydrazine reacts with a sugar in the form of glucose 1. The product was principally the β-isomer, as shown in Scheme 2.

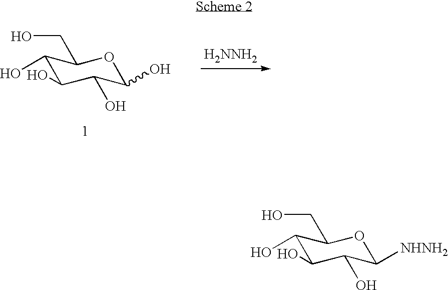

The same reaction was tried using a hydrazide. The representative hydrazide used was adipic dihydrazide 2. Although this could potentially lead to confusion with di-adducts being formed in practice, this was not a problem in interpreting the reaction, as the two hydrazide groups were effectively independent as far as NMR was concerned. The first set of conditions developed to carry-out this reaction were to heat the reactants at 80° C. for 8 hours in a reaction solvent, a 50:50 mixture of water and acetonitrile (Scheme 3). This resulted in a near quantitative yield of the hydrazide adduct 3. It was also found that the reaction could be performed in water/DMSO.

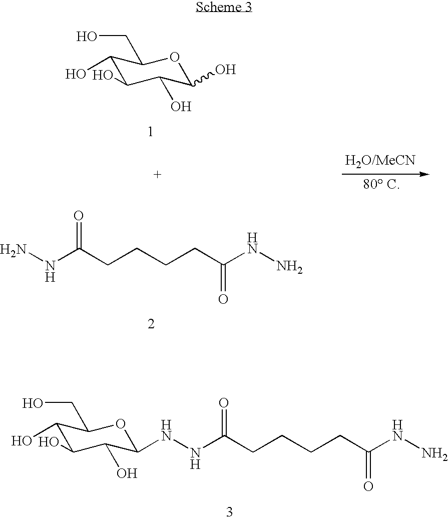

As a result of very high water solubility of both the reactants and the products, purification of the product could not be achieved by any of the usual chemical techniques. However, gel filtration chromatography did allow separation of the desired product from the two starting materials and the di-adduct (glucose-dihydrazide-glucose). As this is a technique that relies on separation based on the size of the molecules, it is most difficult with a monosaccharide such as glucose. Despite this, the desired glucose adduct was purified in a yield of 13% from a reaction using a ten-fold excess of adipic dihydrazide to reduce the amount of the di-glucose adduct formed. The yield of the reaction was undoubtedly much greater than 13%, but this yield reflects the amount of material recovered pure from the column.

In order to be able to obtain the desired adduct cleanly it was necessary to recrystallise the adipic dihydrazide (from water-acetonitrile) prior to use. This was as the small amount of the diadipate impurity 4 (shown in Scheme 4) present in the commercial adipic dihydrazide was concentrated by the gel filtration column to run very close to the desired product. Clearly this is a much bigger problem when an excess of the adipic dihydrazide 2 is used.

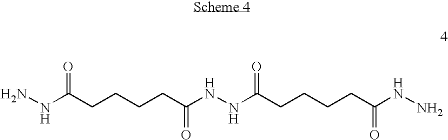

As the purification of the glucose adipic dihydrazide adduct was particularly difficult, the formation of adducts with other sugars was examined. As an example of a disaccharide with an internal acetal linkage, the reaction with α-D-lactose 5 was examined. The reaction conditions developed for glucose were used without further modification. NMR indicated that >90% reaction had occurred.

This selectivity of reaction for an anomeric position bearing a hydroxyl group is important because it means that sugar conjugates can be made without the risk of decomposition (hydrolysis) of the saccharide. It also implies that the mechanism of reaction is via the open chain sugar 7 (Scheme 5.)

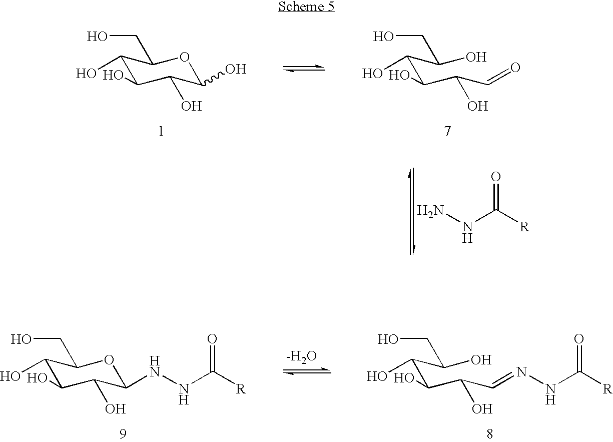

In order to isolate the desired mono-adduct the reaction was performed with a ten-fold excess of the dihydrazide, adipic dihydrazide 2. This led to the desired mono-adduct 6 in an isolated yield of 73% after being chromatographed twice by gel filtration (Scheme 6.) Again the β-isomer was the major isomer, in a similar ratio. The greater difference in size between the product and adipic dihydrazide in this case compared to glucose led to a much greater isolated yield of the product. As stated previously, this improvement in isolated yield was due to an easier isolation rather than a difference in the reaction.

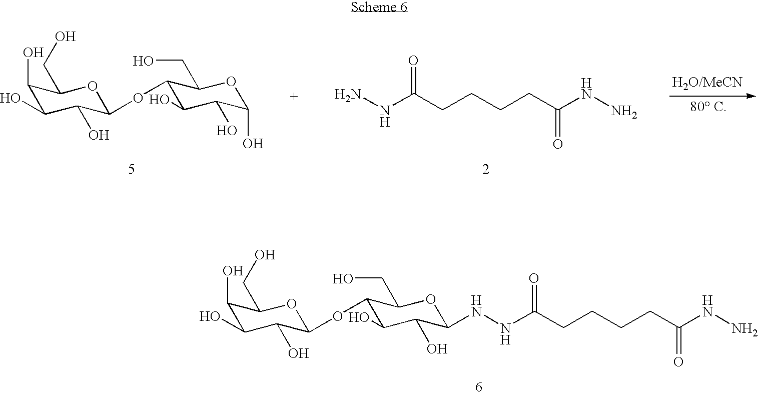

The next saccharide structural type to be investigated was an amino-sugar, using glucosamine 10 (as the hydrochloride) as an example. In this case the reaction was complete within 6 hours, and probably considerably sooner. This was presumably due to acid catalysis, as the starting sugar was present as the hydrochloride salt. Again the β-isomer 11 was the major to a similar degree (Scheme 7.)

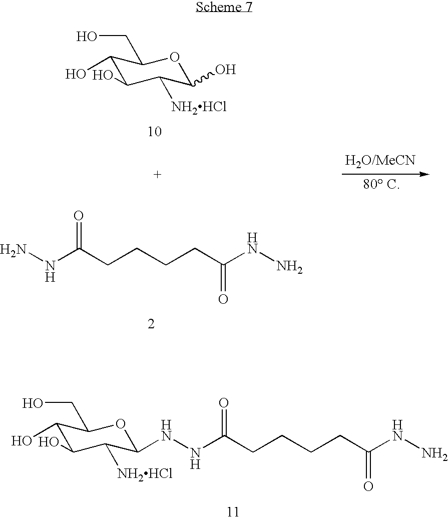

The final saccharide structural type to be examined was an N-acetylamino sugar, using N-acetyl glucosamine 12 as an example. In this case the standard conditions only gave a conversion of about 12% after 6 hours. Clearly the reaction was much slower in this case.

Protic acid would clearly have accelerated the reaction, but in order to maintain selectivity, mild acidic conditions were required. This was achieved by performing the reaction in a formate buffer adjusted to pH 4.75. Heating at 80° C. for 8 hours resulted in effectively complete reaction (Scheme 8.) The fact that equilibrium had been reached was confirmed by continuing the reaction for another 9 hours, at which point the NMR showed no change. Again under these conditions the β-isomer 13 was major to an approximately similar extent.

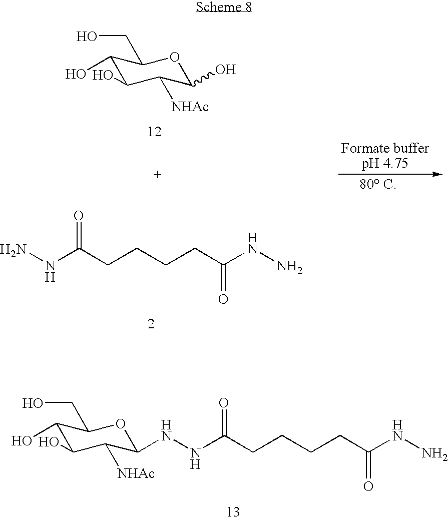

With conditions established that allowed a range of saccharide structural types to be linked via a hydrazide, the stability of this moiety to coupling of the distal hydrazide to a 2-hydroxybenzaldehyde was examined. This is usually achieved by reaction in a buffer in the pH range 3.5-4.5. Given the importance of maintaining the already synthesised hydrazide link and hence achieving selectivity, the initial conditions were chosen at the limits of this range. Thus, to establish conditions for hydrazone formation, a representative benzaldehyde (2-hydroxy-4-methoxybenzaldehyde 14) was reacted with adipic dihydrazide 2 in a mixture of acetonitrile and 100 mM formate buffer (pH 4.75) at room temperature. The reaction was followed by NMR, which showed the extent of reaction increasing with time (FIG. 1.) The estimates of % reaction were approximately ±5%.

The reaction resulted in a mixture of geometrical isomers that was not easy to interpret. To study this in a simpler system the reaction was repeated using acetic hydrazide 15. This resulted in a 2:1 mixture of geometrical isomers (either E and Z hydrazones 16 or amide rotamers) after 1 day at room temperature (Scheme 9.)

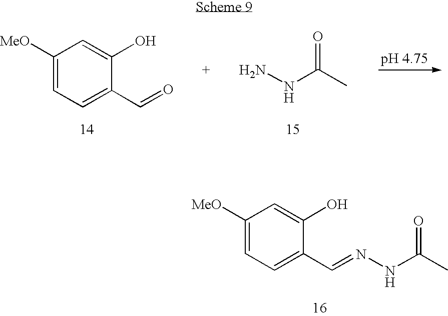

Having established mild conditions for hydrazone formation they were then applied to a sugar hydrazide adduct. Reacting the glucose-adipic dihydrazide 3 adduct for 24 hours resulted in essentially complete conversion of the benzaldehyde 14 to the desired sugar-hydrazone adduct 17 which was formed as an approximate 2:1 mixture of geometrical isomers (Scheme 10.) However, it could be seen that there was (at most) only a trace of glucose 1 present indicating that the original animal was stable to the reaction conditions.

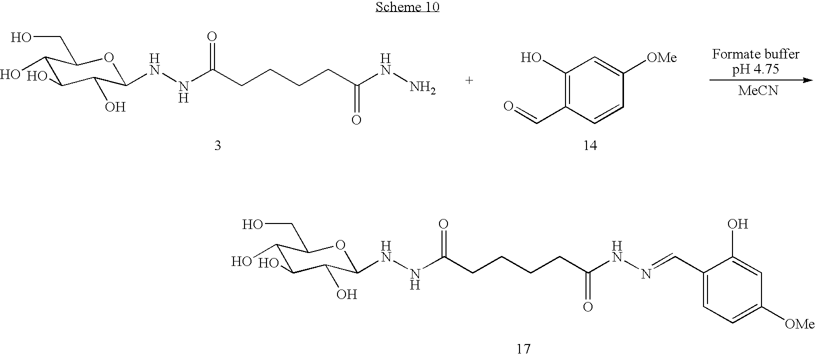

This established that a sugar could be linked to a model of the standard linker (e.g. those disclosed in WO03/087824) through the anomeric position. Although the same type of reaction was involved in each of the two reactions used, the difference in reactivity of an animal and imine type allowed essentially complete selectivity to be achieved.

In order to demonstrate the flexibility of this chemistry, a conjugate of the sugar glucuronic acid 18 and a dihydrazide, adipic dihydrazide 2, through the anomeric position was made. The standard conditions gave a complex mixture of products, though the desired product 19 was present. As it was thought that heating to 80° C. might be causing a problem the reaction was performed in water at 50° C. After 5 hours, NMR analysis indicated that about 50% conversion had occurred (Scheme 11.)

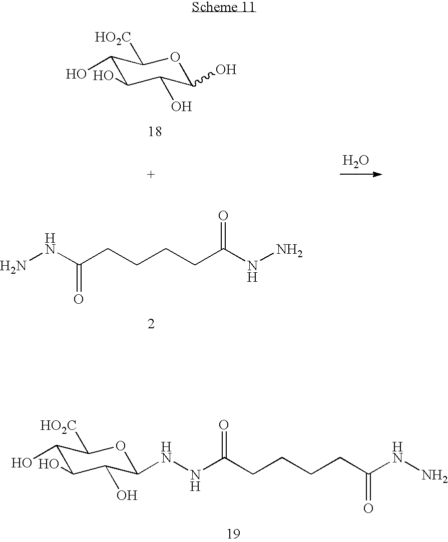

### Model Peptide Conjugation

In order to assess the reactivity of the hydrazide modified sugars in a conjugation reaction with a linker group, a lysine containing model tripeptide 20 was synthesised by solid phase chemistry and N-terminal acetylated. The peptide was then acylated on its lysine side chain with AmLinker (N-hydroxysuccinimide ester) 21 and used as a mimic of a carrier protein 22 (Scheme 12). Amlinker is a linker of the type disclosed in WO03/087824, and was prepared by the method disclosed therein.

The mono- and di-saccharide hydrazide modified sugars 3 and 6 (glucose and lactose respectively) were reacted with AmLinker-peptide 22 under a variety of conditions, to establish optimal reaction conditions for saccharide conjugation and produce conjugates 23 and 24 (Scheme 13). DMSO concentration, solvent pH and molar equivalents were all varied with each sugar hydrazide. A feature of the “AmLinker” technology is that formation of a hydrazone bond between the benzaldehyde function of the linker and a range of hydrazides, results in a reversible absorbance change enabling the forward and reverse reactions to be monitored in situ and quantified in real-time. Thus all reactions were initially performed in a 96-well microtitre plate and monitored by UV spectroscopy (Scanning between 250 nm and 375 nm) (FIGS. 2-4) and by HPLC-coupled mass spectrometry (LC-MS) to identify the correct mass of the conjugates produced.

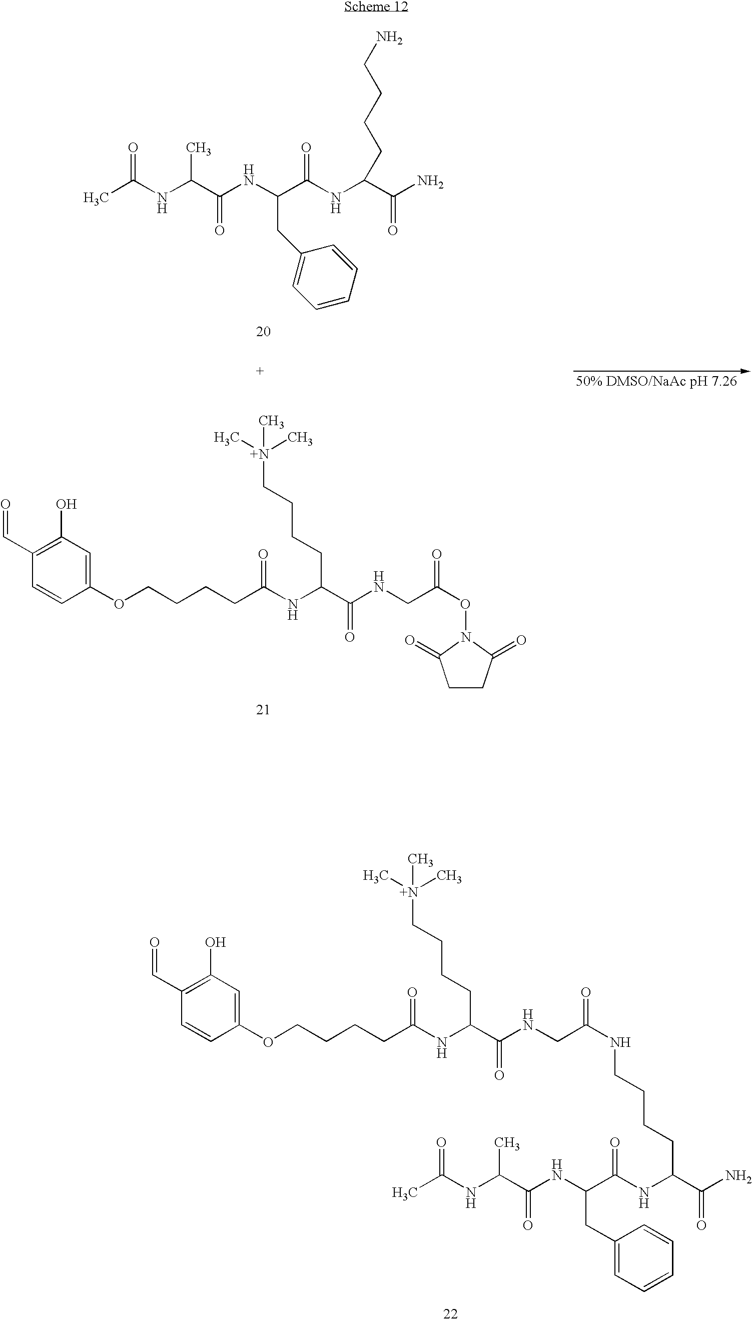

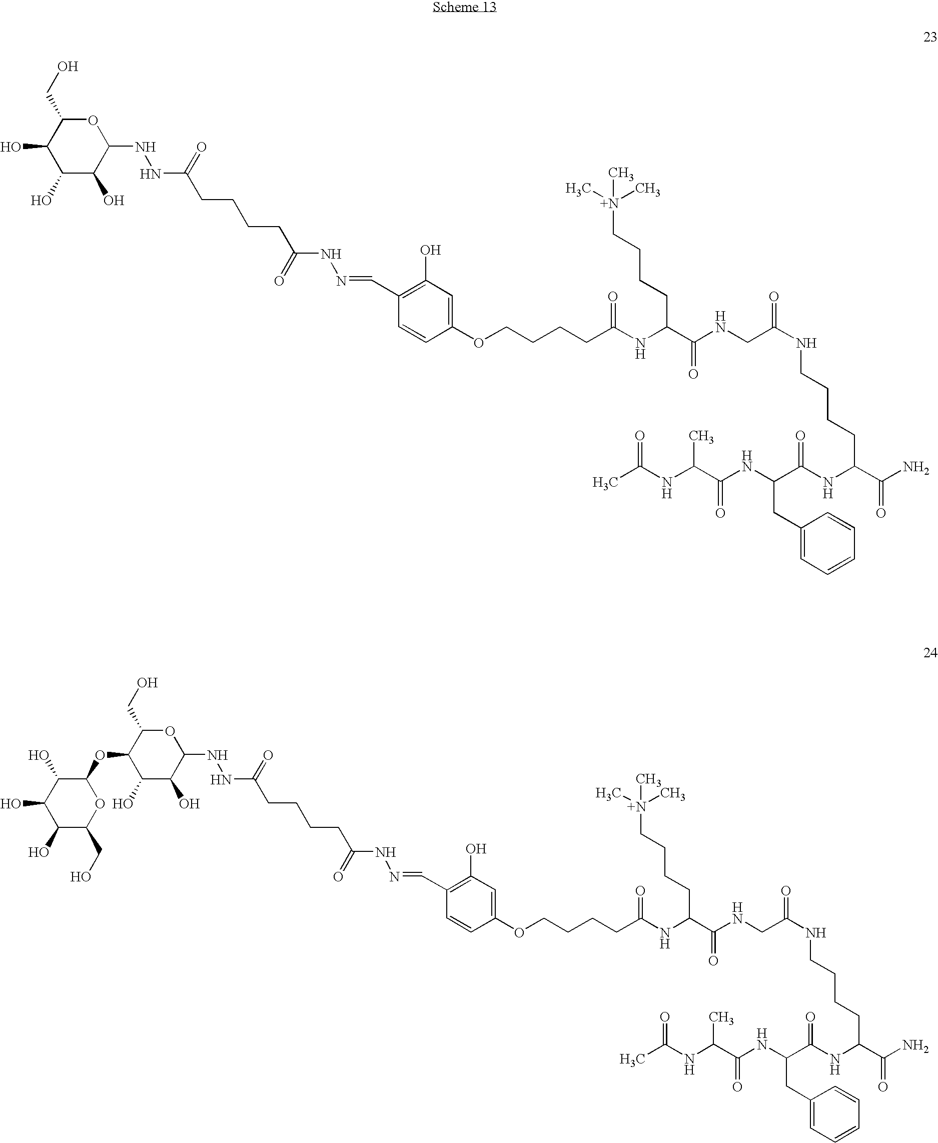

From the graphs in FIGS. 2, 3 and 4, the peak at 318 nm, seen increasing over time, is indicative of the formation of conjugate 24. It is clear that the optimal reaction conditions are pH 4, using 5-10 molar equivalents of hydrazide over the linker, with 10-20% DMSO present. Both conjugates 23 and 24 were resynthesised using the optimal conditions described and the formation of the conjugate monitored by UV at 318 nm.

As can be seen from the graph (FIG. 5), production of the model peptide conjugates 23 and 24 using the optimal conditions proceeded smoothly with the reaction reaching an end point after 5-6 hours. When analysed by LC-MS the conjugates produced gave the correct mass and were of single peak purity, indicating successful conjugation of the sugars to the model peptide (i.e. formation of a sugar-hydrazide-linker-carrier, where the carrier is the model peptide).

### Carrier Protein Conjugation

Having successfully produced model peptide-sugar conjugates and optimised the reaction conditions, the next step was to produce the more complex protein-sugar conjugates. In this case the protein being used was bovine serum albumin (BSA), which is often used as a carrier protein for experimental conjugate vaccines. BSA has a molecular weight of approximately 66 kDa and possesses 60 amine groups, although about only half of these are solvent accessible and amenable for conjugation.

In the same way that the model peptide was modified with AmLinker, BSA was derivatised by acylation with the N-hydroxysuccinimide ester of the linker to produce a BSA-AmLinker derived carrier protein 29 (Scheme 14).

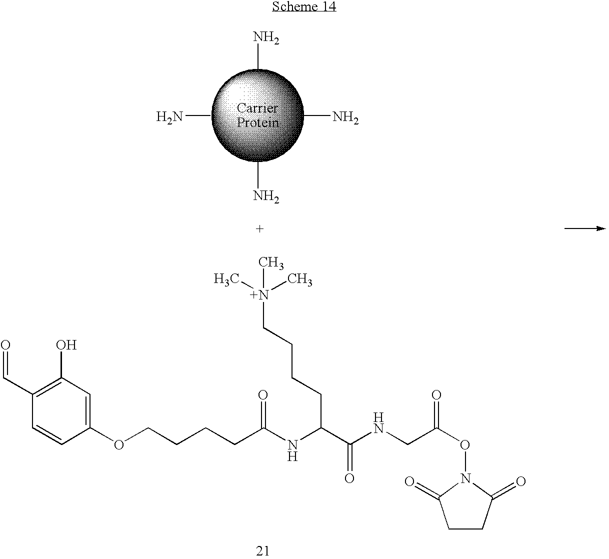

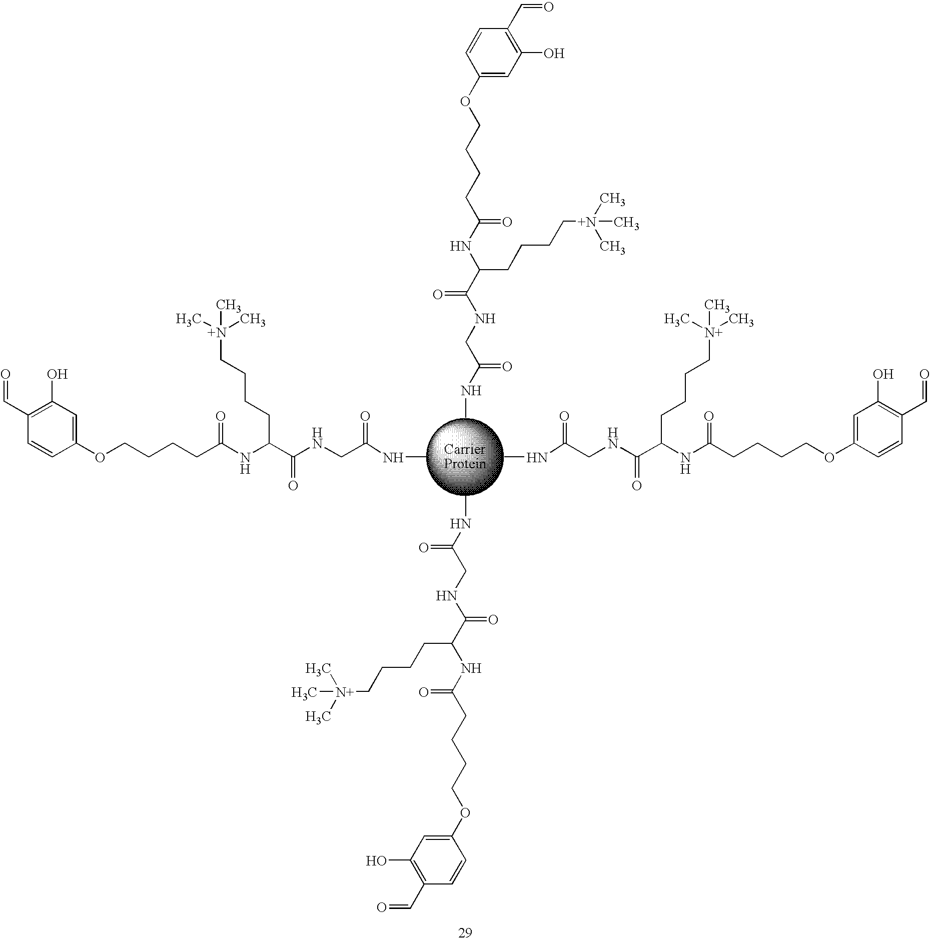

Using the optimal reaction conditions elucidated previously, conjugation reactions were attempted with the AmLinker modified BSA and hydrazide sugars 3 and 6 to produce BSA-sugar conjugates 25 and 26. The process was monitored as before, by UV at 318 nm and gave results very similar to those obtained for the production of the model peptide conjugates 23 and 24 (FIG. 6).

Characterisation of the conjugates was accomplished by gel electrophoresis, which was employed to give an estimation of molecular weight. FIG. 7 shows the slight increase in molecular weight between BSA and conjugates 25 and 26. The sugar-hydrazide-linker-carrier is formed.

### Polysaccharide Epitope Carrier Protein Conjugate

Having successfully demonstrated the concept of controlled conjugation of hydrazide derivitised mono- and disaccharides to a model carrier protein, through AmLinker, a more complex and biologically relevant carbohydrate was used.

There are some commercially available complex carbohydrates that are deemed “tumour associated antigens”, which have been highlighted as possible candidates for vaccination to treat certain cancers. Of particular interest is the blood group related antigen Lewis Y (Le^{y}; Scheme 15). Le^{y} is a carbohydrate specificity belonging to the A, B. H Lewis blood group family that is over-expressed on many carcinomas, including ovary, pancreas, prostate, breast, colon and non-small cell lung cancers. Monoclonal antibodies (mAb) specific for Le^{y} are commercially available and are useful for determining whether the structural conformation of the Le^{y} is retained during the conjugation process, since the Le^{y} mAb will only recognise the native structure.

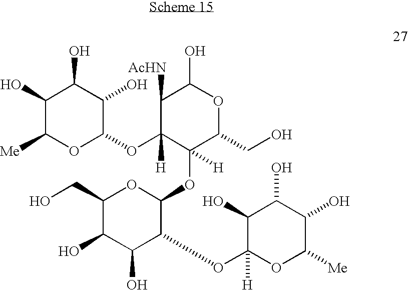

As the Le^{y} saccharide has a 2-acetylamino group the conditions developed for N-acetyl glucosamine would form the basis of the reaction. Trial reactions on small scale showed that with the very small volume of solvent needed it was impossible to prevent the reaction becoming dry as water condensed up the reaction vessel. This could have been addressed by diluting the reaction, but it was decided to overcome the problem by lowering the temperature. Trial reactions showed that at 30° C. the reaction of N-acetyl glucosamine with adipic dihydrazide was essentially complete after 5 days. The reaction was performed at this temperature.

The Lewis Y tetrasaccharide 27 and 10 equivalents of adipic dihydrazide 2 were heated in pH 4.75 formate buffer for 6 days at which point TLC (SiO₂, MeOH) indicated that the reaction was essentially complete. MS showed m/z of 832.2 (M+H⁺) and 854.2 (M+Na⁺), which correspond to the product 28. NMR of the crude reaction mixture indicated that there was one major product. Gel filtration chromatography gave the desired product sugar hydrazide adduct 28 in an isolated yield of 45% (Scheme 16).

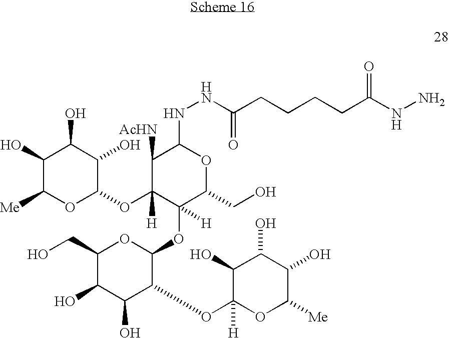

Conjugation of 28 to AmLinker-BSA 29 was carried out and monitored by UV at 318 nm (FIG. 8) as stated previously for conjugates 25 and 26, with the exception that only 3 molar equivalents of 28 were used over the linker.

After purification of the product by diafiltration, the Le^{y}-BSA conjugate 30 [sugar(polysaccharide epitope)-hydrazide-linker-carrier] was initially characterised by gel electrophoresis to assess molecular weight and loading (FIG. 9). The gel in FIG. 9 clearly shows the BSA-Le^{y} conjugate 30 has an increased molecular weight of approximately 25 KDa when compared to the unmodified BSA protein. Modification of the BSA with one AmLinker molecule and a Le^{y} sugar, would lead to an increase of a little over 1 KDa. Thus a molecular weight increase of 25 KDa would suggest a loading of ˜22-24 molecules of Le^{y} on every molecule of BSA.

To further characterise the conjugate, binding assays were performed using a Le^{y} specific mAb to demonstrate that the Le^{y} saccharide was conjugated in a selective manner and remained structurally unperturbed. In the first experiment, a Western Blot of a gel similar to that in FIG. 9 was carried out and exposed to the Le^{y} specific mAb. After exposure of the blot, it was clear that the mAb recognised the BSA-Le^{y} conjugate only and not the BSA or the BSA-AmLinker intermediate (FIG. 10).

To confirm that the Le^{y} specific mAb recognised only the Le^{y} and not the BSA or linker, an ELISA immunoassay was performed, whereby the BSA-Lewis Y conjugate 30 was treated with 1N hydrochloric acid (HCl) and then re-purified by ultra-diafiltration. Since Amura's linker technology is reversible, treatment with acid will remove any conjugated molecule while leaving the BSA-AmLinker preparation intact. Thus, the Le^{y} specific mAb should no longer recognise this acid treated sample after removal of the Le^{y}. The 4 samples (shown in FIG. 11) were added to column 1 of a microtitre plate and double-diluted in PBS across to column 11, column 12 was left blank to provide a negative control. After exposure to the Le^{y} specific mAb, only the BSA-Le^{y} conjugate 30 displayed binding, while the other samples, including the acid treated BSA-Le^{y} conjugate, were not recognised by the mAb, as quantified by absorbance readings at 490 nm (FIGS. 11 & 12).

Thus, the Le^{y} saccharide was conjugated in a selective manner and retained its natural structure after conjugation.

### Experimental Methods

General Biochemistry. All reagents were of the highest commercially available quality and were used as received. Unless otherwise stated all chemicals and biochemicals were purchased from the Sigma Chemical Company (Poole, Dorset, UK). Unless otherwise stated, routine protocols were carried out at room temperature and kinetic experiments at 25° C. Absorbance measurements were carried out in flat-bottomed 96-well plates (Spectra; Greiner Bio-One Ltd., Stonehouse, Gloucestershire, U.K), using a SpectraMax PLUS384 plate reader (Molecular Devices, Crawley, U.K). SOFTmax Pro 3.1.2 software was used for data collection and handling (Molecular Devices). All spectra were collected at a resolution of 2 nm. Gel and membrane images were captured using a Hewlett Packard C7710A scanner employing HP Precision ScanPro 3.02 software on default settings.

General Chemistry. All solid phase synthesis was performed using an “Fmoc/tBu” procedure (Atherton, E., and Sheppard, R. C. (1989) Solid Phase Peptide Synthesis: A Practical Approach, IRL Press, Oxford.) invoking standard solid phase synthesis resin washing protocols. Standard Fmoc amino acids were obtained from Chem-Impex International (Wood Dale, Ill., USA) and Merck Biosciences (Nottingham, UK) with the exception of Fmoc N-ε-trimethyllysins, which was purchased from Bachem UK Ltd. (St. Helens, UK). 2-Chlorotrityl-resin (Product 04-12-2800) was obtained from Merck Biosciences (Nottingham, UK). PS-carbodiimide resin was obtained from Argonaut Technologies (Muttenz, Switzerland). General reagents were purchased from Sigma-Aldrich Chemical Company (Poole, Dorset, UK) unless stated otherwise. Adipic acid dihydrazide was recrystallised from water-acetonitrile prior to use. All solvents were purchased from Romil (Cambridge, UK). Solid phase syntheses were performed manually in a polypropylene syringe fitted with a polypropylene frit to allow filtration under vacuum. Analytical HPLC was performed on Agilent 1100 series instruments including a G1311A quaternary pumping system, with a G1322A degassing module and a G1365B multiple wavelength UV-VIS detector. Data were collected and integrated with Chemstation 2D software. The analyses were performed on a Zorbax, 5 μm, C8 reverse phase column (150×4.6 mm i.d.; Agilent), at a flow rate of 1.5 mL/min., monitoring at 215 and 254 nm. Eluents used were (A) 0.1% trifluoroacetic acid in water and (B) 90% acetonitrile/10% eluent A and used to run a gradient starting at 10% B, increasing to 90% B over 7 min, holding for 1 min, returning to 10% B over 1 min and then remaining at initial conditions for a further 4 min. to allow column re-equilibration. Compounds were purified by semi-preparative HPLC, using a Jupiter C4 reverse phase column (250×10 mm i.d.; Phenomenex) at a flow rate of 4 mL/min., using the equipment and eluents described above. The molecular weight of compounds was determined on an Agilent 1100 series LC/MSD electrospray mass spectrometer.

SDS-PAGE, Western blot and ELISA analysis. The SDS-PAGE and Western blot components were obtained from Invitrogen, Paisley, U.K. For denatured protein analysis, samples were routinely analysed by SDS-PAGE using the NuPAGE system employing 4-12% bis-tris NuPAGE gels and unless otherwise stated, all gels were run in MES or MOPS running buffer. The POWEREASE system was used to carry out electrophoresis employing protocols embedded in the unit's software. Proteins were visualised with SimplyBlue stain or SilverExpress stain kit according to manufacturers' protocols. After staining gels were dried using DryEase gel drying solution and cellophane membranes.

For Western blot analysis of conjugates, the proteins were transferred from the gel onto PVDF membrane using the manufacturers reagents and protocols (Invitrogen). The PVDF membranes were blocked by gentle agitation in 50 mL phosphate buffered saline containing 1% Tween 20 (PBST; Sigma) plus 1% (w/v) casein for 60 min. The recovered membranes were washed three times, by gentle agitation, in 50 mL PBST for 5 min per cycle. Following this, the membranes were incubated in 30 mL PBST containing 1:100 dilution of mouse IgM Le^{y} monoclonal antibody (Alexis Corporation # SIG317) for 60 min. After washing as before, the membranes were incubated in 30 mL PBST containing 1:1000 dilution goat anti-mouse IgM, HRP conjugated antibody (Alexis Corporation # A90-101P) for 60 min, washed as before in PBST and allowed to partially drip-dry. Regions of peroxidase activity were visualized by addition of a 3,3′,5,5′-tetramethylbenzidine (TMB) liquid substrate (Sigma) onto the static membrane. After appropriate exposure, the membrane was recovered, washed in water and air dried prior to scanning, analysis and storage.

ELISA assays were performed in immulon 2HB 96-well plates (Thermo Labsystems). Samples were introduced in PBS buffer and incubated overnight at 37° C. Washing was performed 3 times with 200 μL PBST and the plate blocked with 100 μL of PBST containing 1% casein (w/v) for 60 min. Primary and secondary antibodies (100 μL) were the same as those used for the Western Blot analysis, both being incubated for 60 min at 37° C. with a PBST wash step in between. After a final PBST wash, peroxidase activity was visualized by addition of 100 μL o-phenylenediamine (OPD; Sigma) and the reaction quenched with 100 μL of 0.1 M sulphuric acid. Quantitation was by absorbance at 490 nm.

Diafiltration. Routinely, Amicon Ultra-4 (<4 mL) or Ultra-15 (<15 mL) centrifugal filter units (10,000 mwco; Millipore, Watford, U.K.) were used for diafiltration. Each cycle consisted of diluting the protein sample approximately forty-fold with exchange buffer and concentrating the sample by centrifugation back to its original volume. Cycles were repeated as required for quick and highly efficient equilibration/washing of protein samples. Routinely, six cycles were completed for each diafiltration step.

General conjugation procedure. Unless otherwise stated, conjugation reactions were carried out at room temperature in sodium formate (0.1M; pH 4) containing between 10% and 30% DMSO, using 3-5 molar equivalents of sugar hydrazide over the linker or AmLinker-BSA (29). The reactions were monitored by UV at 318 nm and allowed to run until deemed completed (by the UV profile), which in general was 5-6 hours.

### Chemistry

### 5-[N′-(3,4,5-Trihydroxy-6-hydroxymethyl-tetrahydro-pyran-2-yl)-hydrazinocarbonyl]-pentanoic hydrazide 3

A mixture of glucose 1 (90 mg, 0.5 mmol) and adipic dihydrazide 2 (870 mg, 5 mmol) in water (5 ml) and acetonitrile (5 ml) was heated at 80° C. After 8 hours the mixture was evaporated under reduced pressure, water (2 ml) was added and the mixture evaporated under reduced pressure. Gel filtration chromatography (Bio-Gel P-2 Gel, extra fine, 0.02 M ammonium bicarbonate) was performed twice to give 5-[N′-(3,4,5-trihydroxy-6-hydroxymethyl-tetrahydro-pyran-2-yl)-hydrazinocarbonyl]-pentanoic hydrazide 3 (22 mg, 13%). ¹H NMR (D₂O) δ: 3.96 (d, 1H, J=9.0 Hz), 3.77 (dd, 1H, J=12.2, 2.1 Hz), 3.58 (dd, 1H, 12.2, 5.8 Hz), 3.38 (t, 1H, J=9.1 Hz), 3.28 (ddd, 1H, J=9.8, 5.9, 2.3 Hz), 3.22 (t, 1H, J=9.3 Hz), 3.14 (t, 1H, J=9.0 Hz), 2.11 (m, 4H), 1.47 (m, 4H). MS m/z; 337.2 (M+H⁺), 359.2 (M+Na⁺); Exact mass calcd for C₁₂H₂₄N₄O₇ (MH+): 337.1718, found 337.1710 (δ −2.19 ppm).

### 5-{N′-[3,4-Dihydroxy-6-hydroxymethyl-5-(3,4,5-trihydroxy-6-hydroxymethyl-tetrahydro-pyran-2-yloxy)-tetrahydro-pyran-2-yl]-hydrazinocarbonyl}-pentanoic hydrazide 6

A mixture of α-D-lactose monohydrate 5 (180 mg, 0.5 mmol) and recrystallised adipic dihydrazide 2 (870 mg, 5 mmol) was heated in water (5 ml) and acetonitrile (5 ml) at 80° C. After 8 hours the mixture was evaporated under reduced pressure, water (2 ml) added and the mixture evaporated under reduced pressure. Gel filtration chromatography (Bio-Gel P-2 Gel, extra fine, 0.02 M ammonium bicarbonate) was performed twice to give 5-{N′-[3,4-dihydroxy-6-hydroxymethyl-5-(3,4,5-trihydroxy-6-hydroxymethyl-tetrahydro-pyran-2-yloxy)-tetrahydro-pyran-2-yl]-hydrazinocarbonyl}pentanoic hydrazide 6 (182 mg, 73%). ¹H NMR (D₂O) for β-isomer δ: 4.35 (d, 1H, J=7.9 Hz), 4.03 (d, 1H, J=9.0 Hz), 3.87 (dd, 1H, J=12.2, 2.1 Hz), 3.83 (d, 1H, J=3.3 Hz), 3.75-3.50 (m, 7H), 3.45 (m, 2H), 3.25 (t, 1H, J=9.0 Hz), 2.14 (m, 4H), 1.51 (m, 4H). MS m/z; 499.2 (M+H⁺), 521.2 (M+Na⁺), 1019.3 (2M+Na⁺); Exact mass calcd for C₁₈H₃₄N₄O₁₂ (MH+): 499.2246, found 499.2252 (δ +1.25 ppm).

### 5-[N′-(3-Amino-4,5-dihydroxy-6-hydroxymethyl-tetrahydro-pyran-2-yl)-hydrazinocarbonyl]-pentanoic hydrazide hydrochloride 11

A mixture of glucosamine hydrochloride 10 (215 mg, 1 mmol) and adipic dihydrazide 2 (174 mg, 1 mmol) in water (1 ml) and acetonitrile (1 ml) was heated at 80° C. for 6 hours. At this point NMR showed that virtually all the glucosamine hydrochloride had been converted to hydrazide. The product 11 was not isolated. ¹H NMR (D₂O) for β-isomer δ: 4.31 (d, 1H, J=9.7 Hz), 3.83 (dd, 1H, J=12.2, 1.9 Hz), 3.67-3.60 (m, 2H), 3.41 (m, 1H), 3.33 (t, 1H, J=9.3 Hz), 2.92 (td, 1H, J=10.1, 3.2 Hz), 2.19 (m, 4H), 1.51 (m, 4H). MS m/z; 336.2 (M+H⁺), 671.3 (2M+H⁺).

### 5-[N′-(3-Acetylamino-4,5-dihydroxy-6-hydroxymethyl-tetrahydro-pyran-2-yl)-hydrazinocarbonyl]-pentanoic hydrazide 13

A mixture of N-acetyl glucosamine 12 (221 mg, 1 mmol) and adipic dihydrazide 2 (174 mg, 1 mmol) in pH 4.75 formate buffer (1 ml) was heated at 80° C. After 8.5 hours NMR showed that approximately 90% of the N-acetyl glucosamine had been converted to hydrazide. The product 13 was not isolated. ¹H NMR (D₂O) for β-isomer δ: 4.12 (d, 1H, J=9.6 Hz), 3.82 (brd, 1H, J=12 Hz), 3.64 (t, 1H, J=9.9 Hz), 3.64 (brdd, 1H, J=12.2, 4.6 Hz), 3.47, (m, 1H), 3.33 (m, 1H), 2.15 (m, 4H), 1.94 (s, 3H), 1.50 (m, 4H). MS m/z; 378.3 (M+H⁺), 400.2 (M+Na⁺), 777.3 (2M+Na⁺).

### 6-Oxo-7-aza-7-(3,4,5-trihydroxy-6-hydroxymethyl-tetrahydro-pyran-2-ylamino)-heptanoic acid (2-hydroxy-4-methoxy-benzylidene)-hydrazide 17

A mixture of 5-[N′-(3,4,5-Trihydroxy-6-hydroxymethyl-tetrahydro-pyran-2-yl)-hydrazino-carbonyl]-pentanoic hydrazide 3 (8.8 mg, ˜76%, 0.02 mmol) and 2-hydroxy-4-methoxybenzaldehyde 14 (4.6 mg, 0.03 mmol) in pH 4.75 buffer (0.05 ml) and acetonitrile (0.05 ml) was stirred at room temperature for 24 hours. Two drops of saturated sodium hydrogen carbonate were then added to adjust the pH to approximately 9 and the mixture was evaporated under reduced pressure. Water (0.5 ml) was added and the mixture filtered and evaporated under reduced pressure. NMR indicated that essentially all the benzaldehyde had been converted to the adduct 17 and that at most only a trace of glucose had been generated. ¹H NMR (D₂O) for major isomer aromatic region δ: 7.44 (d, 1H, J=8.8 Hz), 6.36 (dd, 1H, J=8.8, 2.3 Hz), 6.31 (d, 1H, J=2.3 Hz). ¹H NMR (D₂O) for minor isomer aromatic region δ: 7.44 (d, 1H, J=8.8 Hz), 6.18 (dd, 1H, J=8.9, 2.4 Hz), 6.12 (d, 1H, J=2.4). MS m/z; 471.2 (M+H⁺), 493.2 (M+Na⁺), 963.3 (2M+Na⁺).

### 6-[N′-(5-Carboxy-pentanoyl)-hydrazino]-3,4,5-trihydroxy-tetrahydro-pyran-2-carboxylic hydrazide 19

A mixture of glucuronic acid 18 (194 mg, 1 mmol) and adipic dihydrazide 2 (174 mg, 1.0 mmol) in water (1 ml) was heated at 50° C. After 5 hours the mixture was cooled to room temperature, diluted with water (4 ml), frozen and lyophilised. NMR showed the reaction to have proceeded to approximately 50%. A separate reaction produced an approximately 80 mol % pure sample of 6-[N′-(5-carboxy-pentanoyl)-hydrazino]-3,4,5-trihydroxy-tetrahydro-pyran-2-carboxylic hydrazide 19 (contaminated with 20 mol % adipic dihydrazide) after gel filtration chromatography (Bio-Gel P-2 Gel, extra fine, 0.02 M ammonium bicarbonate). ¹H NMR (D₂O) for the β-isomer δ: 4.02 (d, 1H, J=9.1 Hz), 3.62 (d, 1H, J=9.8 Hz), 3.45 (t, 1H, J=9.1 Hz), 3.37 (t, 1H, J=9.5 Hz), 3.22 (t, 1H, J=9.1 Hz), 2.15 (m, 4H), 1.51 (m, 4H). MS m/z; 351.2 (M+H⁺), 373.2 (M+Na⁺).

### Alternative preparation of 5-[N′-(3-acetylamino-4,5-dihydroxy-6-hydroxymethyl-tetra-hydro-pyran-2-yl)-hydrazinocarbonyl]-pentanoic hydrazide 13

A mixture of N-acetyl glucosamine 12 (2.2 mg, 0.01 mmol) and adipic dihydrazide 2 (17.4 mg, 0.1 mmol) in pH 4.75 formate buffer (0.1 ml) was heated at 30° C. After 2 days pH 4.75 formate buffer (0.05 ml) was added. After a further 3 days the mixture was evaporated under reduced pressure. NMR showed the product to be approximately 95% 5-[N′-(3-acetylamino-4,5-dihydroxy-6-hydroxymethyl-tetrahydro-pyran-2-yl)-hydrazinocarbonyl]-pentanoic hydrazide 13 as an approximate 85:10 ratio of β:α anomers. The product was not isolated.

### N-[2-(N′-Acetyl-hydrazino)-5-[4,5-dihydroxy-6-hydroxymethyl-3-(3,4,5-trihydroxy-6-methyl-tetrahydro-pyran-2-yloxy)-tetrahydro-pyran-2-yloxy]-6-hydroxymethyl-4-(3,4,5-trihydroxy-6-methyl-tetrahydro-pyran-2-yloxy)-tetrahydro-pyran-3-yl]-5-hydrazinocarbonylpentamide 28

### Lewis-Y Tetrasaccharide-Adipic Dihydrazide Adduct

A mixture of Lewis-Y tetrasaccharide 27 (3 mg as supplied by Sigma, 0.0044 mmol) and adipic dihydrazide 2 (7.7 mg, 0.044 mmol) in pH 4.75 formate buffer (0.1 ml) was heated at 30° C. After 6 days the mixture was diluted with water (0.1 ml), frozen and lyophilised. Gel filtration chromatography (Bio-Gel P-2 Gel, extra fine, 0.02 M ammonium bicarbonate) and lyophilisation gave the Lewis-Y tetrasaccharide-adipic dihydrazide adduct 28 (1.66 mg, 45%). ¹H NMR (D₂O) major isomer δ: 5.15 (d, 1H, J=3.5 Hz), 4.97 (d, 1H, J=3.9 Hz), 4.76 (brq, 1H, J=6.0 Hz), 4.37 (d, 1H, J=7.8 Hz), 4.13 (d, 1H, J=4.6 Hz), 4.11 (brs, 1H), 3.86 (d, 1H, J=10.3 Hz), 3.80-3.40 (m, 19H), 3.30 (m, 1H), 2.25-2.05 (m, 4H), 1.89 (s, 3H), 1.55-1.40 (m, 4H), 1.13 (d, 3H, J=8.6 Hz), 1.10 (d, 3H, J=6.6 Hz). MS m/z; 832.3 (M+H⁺), 854.3 (M+Na⁺); Exact mass calcd for C₃₂H₅₇N₅O₂₀ (MNa+): 854.3495, found 854.3470 (δ −2.92 ppm).

### 2-[2-(2-Acetylamino-propionylamino)-3-phenyl-propionylamino]-6-amino-hexanoic acid amide 20

### Model Tripeptide

20 was synthesised manually using Fmoc/tBu protection strategy on TGR resin (0.25 g, 0.05 mmol), (substitution: 0.2 mmol/g). Fmoc-Lys(ffa)-OH, Fmoc-Phe-OH and Fmoc-Ala-OH were coupled using an HBTU/HOBt method with DMF as the solvent and 3 equivalents of amino acid and coupling reagents with respect to the loading of the resin. The Fmoc group was removed by a 15 min treatment with 20% piperidine in DMF. Prior to cleavage the N-terminal amine was acetylated with acetic anhydride/N-methyl morpholine (10 and 5 equivalents respectively) in DMF for 1 hour. Final cleavage from the resin was performed with TFA/water (95/5) for 75 mins. The resin was removed by filtration and the pooled filtrate was concentrated by sparging with nitrogen. The crude product was precipitated and washed with cold methyl tert-butyl ether, before being re-dissolved in 30% (aq) acetonitrile and lyophilised. Deprotection of the trifluoroacetyl protecting group on the lysine side chain was effected using 5% (w/v) potassium carbonate (containing 5% DMSO) at pH 10, for 24 hours. Finally, the compound was purified by semi-preparative RP-HPLC, the pure fractions pooled and lyophilised once more to yield an white solid. Yield: 11 mg, 0.027 mmol, 54%. ESI-MS m/z: 406.2 (calc. for M+H⁺ 406.49). HPLC retention time: 3.05 mins.

### 5-(4-Formyl-3-hydroxy-phenoxy)-pentanoic acid 2,5-dioxo-pyrrolidin-1-yl ester 21

### Amlinker N-Hydroxysuccinimide Ester

AmLinker (N-hydroxysuccinimide ester) 21 was prepared as described in WO03/087824. The compound was purified by semi-preparative RP-HPLC, the pure fractions pooled and lyophilised once more to yield an off white solid. Yield: 35 mg, 0.075 mmol, 39%. ESI-MS m/z: 466.2 (calc. for M+H⁺ 466.26). HPLC retention time: 3.75 mins. The purified intermediate was dissolved in DMF (2 mL) and added to a stirred solution of PS-carbodiimide (288 mg, 0.375 mmol) in dichloromethane (10 mL). The mixture was stirred for 20 mins before the addition of N-hydroxysuccinimide (9 mg, 0.075 mmol) dissolved in DMF (1 mL). The reaction was then stirred at room temperature and monitored by HPLC until completion (5 hours). The resin was removed by filtration, the solvent removed in vacuo and the compound used without further purification. Yield: 38 mg, 0.068 mmol, 90%. ESI-MS m/z 563.3 (calc. for M+H⁺ 563.3). HPLC retention time: 4.16 mins. {5-[({5-[2-(2-Acetylamino-propionylamino)-3-phenyl-propionylamino]-5-carbamoyl-pentylcarbamoyl}-methyl)-carbamoyl]-5-[5-(4-formyl-3-hydroxy-phenoxy)-pentanoylamino]-pentyl}-trimethyl-ammonium 22

### AmLinker-Model Peptide

AmLinker modified model peptide 22 was prepared by stirring 20 (2.75 mg; 6.8 μmol) and 21 (5.7 mg; 10.1 μmol) in 0.1 M sodium acetate (pH 7.25)/DMSO (50/50). After 2 hours the reaction was lyophilised and purified by semi-preparative HPLC. Yield: 1.2 mg, 1.92 μmol, 28%. ESI-MS m/z: 853.4 (calc. for M+H⁺ 853.6). HPLC retention time: 5.48 mins.

### Conjugates (23) and (24).

The glucose 23 and Lactose 24 conjugates were produced by stirring AmLinker-model peptide 22 with sugar hydrazides 3 and 6, using the general conjugation procedure given above. Conjugate 23 ESI-MS m/z: 1171.5 (calc. 1171.64). HPLC retention time: 3.21 mins. Conjugate 24 ESI-MS m/z: 1334.4 (calc. 1334.49). HPLC retention time: 3.11 mins.

### AmLinker-BSA (29)

BSA (2 mg, 29 nmol) was dissolved in 0.1 M sodium acetate (1 mL, pH 7.25) and 500 μL of 15 mM 21 (in 100% DMSO) added, the reaction was stirred at room temperature. The disappearance of free amine was monitored and once complete (˜2-3 h), the reaction mixture was dialyzed against three changes (2 h each) of 2 L 10 mM ammonium bicarbonate, pH 8 and the product analyzed by SDS-PAGE.

### BSA Conjugates (25), (26) and BSA-Lewis Y Conjugate (30).

The glucose 25 and Lactose 26 conjugates were produced by stirring AmLinker-BSA (29) with sugar hydrazides 3 and 6, using the general conjugation procedure given above, while the Lewis Y conjugate (30) was prepared from AmLinker-BSA (29) and Lewis Y hydrazide 28. The conjugates were purified by diafiltration and characterised by SDS-PAGE.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

1. A method of production of a hydrazide modified sugar comprising a step of reacting a sugar with a Hydrazide in a reaction solvent at a pH of between 3 and 5.5, wherein the solvent comprises an aqueous based solvent and an optional polar organic co-solvent.

2. A method according to claim 1 in which the sugar is a polysaccharide or a polysaccharide epitope.

3. A method according to claim 1 or claim 2 in which the polysaccharide epitope is an antigenic determinant derived from a surface molecule from a pathogenic organism.

4. A method according to claim 1 or claim 2 in which the polysaccharide or polysaccharide epitope is a tumour associated antigen.

5. A method according to claim 4 in which the tumor associated antigen is Lewis Y tetrasaccharide.

6. A method according to any preceding claim in which the pH is between 3.5 and 5.

7. A method according to claim 1 in which the reaction solvent includes a buffer solution.

8. A method according to claim 1 in which the aqueous solvent is a buffer solution.

9. A method according to claim 1 in which the hydrazide is present in an amount of up to 50% (by volume) of the total amount of the reaction solvent.

10. A method according to claim 1 in which the hydrazide is a dihydrazide which is a branched or straight chain alkyl of up to 10 carbon atoms having a first hydrazide moiety at one end of the alkyl chain and the second hydrazide moiety at the other end of the chain.

11. A method of production of a polysaccharide epitope carrier protein conjugate comprising the steps of:
(a) reacting a polysaccharide epitope with a hydrazide to form a hydrazide modified polysaccharide epitope;
(b) reacting the hydrazide modified polysaccharide epitope with a linker that has been pre-coupled to a carrier protein.

12. A method according to claim 11 in which the hydrazide in step (a) is a dihydrazide and the product of step (a), the hydrazide modified polysaccharide epitope, includes a further unreacted hydrazide moiety; and step (b) includes the reaction of the further hydrazide moiety with a suitable group on the linker.

13. A method according to claim 11 or 12 in which reaction (a) and/or reaction (b) is performed in a reaction solvent at a pH of between 3 and 5.5, wherein the solvent comprises an aqueous base solvent and an optional polar organic co-solvent.

14. A method according to claim 13 in which the reaction solvent includes a buffer solution which maintains the preferred pH range.

15. A method according to any of claims 12 or 14 in which the linker includes an aldehyde functionality which reacts with the further hydrazide moiety.

16. A method according to any claims 11, 12 or 14 in which the linker is capable of undergoing a specific chemical reaction with both a carrier and the further hydrazide.

17. A method according to any of claims 11, 12, or 14 in which the carrier is a proteinaceous molecule.

18. A method according to any of claims 11, 12 or 14 in which the polysaccharide epitope is Lewis Y tetrasaccharide; the carrier protein is BSA; and the polysaccharide epitope carrier protein conjugate is a synthetic Le^{y}-BSA conjugate.

19. A pharmaceutical composition or a diagnostic composition comprising a polysaccharide epitope carrier protein conjugate made by a method according to claim 11.

20. A vaccine composition comprising a pharmaceutical composition according to claim 19.

21. A method of production of a sugar-dihydrazide-aldehyde adduct comprising the steps of:
(a) producing a hydrazide modified sugar using a method according to claim 1 wherein the hydrazide modified sugar includes a further unreacted hydrazide moiety; and
(b) reacting the further hydrazide moiety with the aldehyde functionality of a linker group.

22. A method according to claim 21 in which reaction (b) is performed in a reaction solvent at a pH of between 3 and 5.5, wherein the solvent comprises an aqueous base solvent and an optional polar organic co-solvent.

23. A method according to claim 22 in which the reaction solvent includes a buffer solution which maintains the preferred pH range.

24. A method according to claim 21 in which the linker undergoes a specific chemical reaction with both the further hydrazide and a carrier.

25. A method according to claim 24 in which the carrier is a proteinaceous molecule.

26. The BSA-AmLinker derived carrier protein substantially as hereinbefore described and referred to by numeral 29 in scheme 14.
